(19) United States
(12) Patent Application Publication
(10) Pub. No.: US 2022/0268462 A1
(43) Pub. Date: Aug. 25, 2022

(54) Multidirectional Light-emitting Apparatus and Method for Sterilizing Air

(71) Applicant: Ron Sasonov, West Hartford, CT (US)

(72) Inventor: Ron Sasonov, West Hartford, CT (US)

(21) Appl. No.: 17/183,089

(22) Filed: Feb. 23, 2021

(51) Int. Cl. F24F 8/22; A61L 2/10

(52) U.S. Cl. CPC F24F 8/22; A61L 2/10; F21Y 2115/10

(57) ABSTRACT

The prior art comprises one lighting set configured to direct light of one or more colors in a specified direction, typically perpendicular to the floor. When the lighting apparatus of the prior art is powered on, all the lighting sources comprising a single lighting set of a lighting apparatus are operable together. All the lighting sources of the prior art, as part of a lighting set in a lighting apparatus, are dimmed together, may change colors together, and are powered ON or OFF together. The preferred invention comprises a lighting apparatus comprising at least two lighting sets directing the light emitted from the lighting sources comprising each lighting set in a different direction in such a way that eliminates or substantially minimizes interference between the light emitted from each lighting set. Each of the lighting sets may be controlled centrally as having two separate control addresses such that each lighting set can selectively be switched ON or OFF and have the brightness and color manipulated by the central controller. Interference among the lights emitted from the two lighting sources, whether constructive or destructive, if not separated through the lighting surfaces designed to control each lighting set's light-emitting direction, may cause a sub-optimal lighting experience. A sub-optimal lighting experience can be characterized as a change in the perceived light color, brightness, or other properties of light as a result of superposition of another proximate light source(s). The preferred invention comprises one of at least two lighting sets, of which the lighting sources comprising the lighting set emit UVC light which has sterilizing properties that are most effective without interference from another lighting set. In the preferred invention the UVC light is emitting in a direction parallel to the floor in a room and the other lighting set is emitting traditional yellow or white light perpendicular to the floor.

### BACKGROUND OF THE INVENTION

A lighting source or a plurality of lighting sources are typically housed in a lighting apparatus directing light in a designated direction(s), often illuminating a room, with the light intensity concentrated at the light source. A lighting apparatus may have one or more lighting sources, such as light bulbs, which are typically powered ON or OFF together and are dimmable together as a collective unit. A user could change the bulbs, the lighting sources affixed to the lighting apparatus to emit different colors. However, if different bulbs were configured to a single fixture, there would be significant interference that the intended color of any one of the pluralities of bulb colors would be perceived as a blend of the bulb colors, due to interference of the surrounding bulbs.

Lighting sources take several forms, including, but not limited to incandescent, fluorescent, and light-emitting diodes (LEDs). LED-based lights have significant advantages over both fluorescent and incandescent light sources for their efficiency, flexibility in creating lighting outcomes, and environmental friendliness. LEDs enable a lighting apparatus to be much more flexible in terms of color configurations emitted from a single lighting apparatus. However, LED's as the lighting sources of a lighting set are controlled together from one circuitry. Multiple LED strips can be adjoined, powered by a driver circuit, and often controlled as one collective unit, though it is possible to configure the control circuitry such that only some of the LEDs are powered ON or emitting a designated light wavelength.

Even still, if a plurality of LED lighting sources may be controlled separately, they still emit light as a single lighting set, characterized by distinct plane, or cumulative direction in which the lights, as a collective unit, are designed to illuminate. Each LED is attached to a base on a different part of the surface area of the light apparatus, though the LEDs of a lighting set are typically close enough to each other such that if they were powered on together interference would exist between the light sources of the set. This interference within the set could cause the intensity, or brightness to be amplified and create one perceived color. If more than one lighting set exists within an apparatus, said interference could also occur between the lighting sets much like it does between the individual sources of a lighting set.

The ability to prevent, or substantially minimize said interference between two or more lighting sets, enables at least two different lighting sets and the lighting sources that comprise each lighting set to emit light in different directions while maintaining their integrity and perceived color, among other benefits. A user who wishes to implement one lighting set emitting UVC light, as an example, which has strong anti-pathogenic properties, may not want to replace a traditional light source in a one-for-one tradeoff in order to benefit from the UVC light. Without the ability to prevent interference of a second lighting from within a lighting fixture, it would not be possible, or be far less effective and desirable to retain a traditional lighting set and a UVC lighting set from a single apparatus as the interference between the lighting sets would create a blended light that perhaps is not as desirable as each individual lighting set.

### BRIEF SUMMARY OF THE INVENTION

Embodiments described herein pertain generally to a multidirectional light-emitting apparatus that may be comprised of at least two lighting sets. Each of the lighting sets emits light in a different direction and may emit a different color light, as characterized by the electromagnetic spectrum. The lighting apparatus may be used in a method for sterilizing air when at least one of the lighting sets is comprised of UV lighting sources which have sterilizing qualities, while minimizing interference from the other lighting set so to maintain a desired ambience as created by the non-UV lighting set.

### BRIEF DESCRIPTION OF THE DRAWINGS

FIG. 1A is a perspective view of the lighting apparatus

FIG. 1B is a top view of the lighting apparatus

FIG. 2 is an exploded view of the lighting apparatus

FIG. 3 is a cross-sectional view of the lighting apparatus showing the lighting sets and their respective lighting surfaces and demonstrates light emitted in two different directions

FIG. 4A is a side view of the top housing component without the second lens attached

FIG. 4B is a bottom view of the top housing component

FIG. 4C is a top view of the top housing component

FIG. 5 is a perspective view of the bottom housing component illustrating a second lighting set in one embodiment

FIG. 6 is a first lens in a preferred embodiment

FIG. 7 is a second lens in a preferred embodiment

FIG. 8 is an illustration of a method for sterilizing air in a room

### DETAILED DESCRIPTION

This disclosure relates to a lighting apparatus comprising a lighting housing and at least two lighting sets. The lighting sets are each configured to at least one lighting surface on at least one of the housing components such that light may be emitted in at least two distinct directions with minimal, if any interference from the other lighting set. Light is emitted from each lighting set through at least a first lens to transmit light from the lighting sources of each lighting set. In a preferred embodiment at least one lighting set is emitted from a second lens.

The lighting apparatus has more than one lighting surface, of which each lighting set, or lighting sets, may be affixed to. The lighting apparatus can have one or more housing components attached together to enclose the lighting sets and surfaces as one lighting apparatus. Each lighting set is affixed to at least one lighting surfaces, distinct from the lighting surface of the other lighting set(s), and each lighting set transmits light through a dedicated lens, such that light is emitted in two different directions, or planes, with minimal interference between the lights emitted from the lighting sources comprising each lighting set.

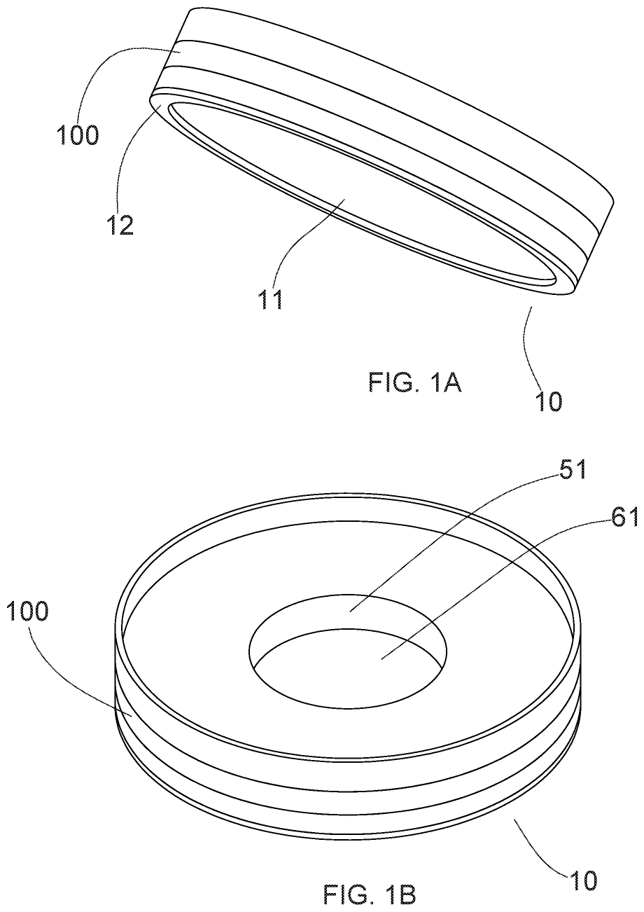

FIG. 1A is a perspective view of a lighting apparatus 10. A first lens 11 is nested above a first lens ridge 12. Light emitted from a first lighting set 31 transmits through the first lens 11. Light emitted from a second lighting set 41 transmits through a second lens 100. In other embodiments, the circular lens 11 could be any shape, not limited to a circular shape. In said embodiments the shape of the housing component(s) would be modified to accommodate the shape of the first lens 11. In said embodiments, the shape of the second lens 100 would change to accommodate the corresponding change in shape of the housing component. In yet another embodiment, the lighting apparatus 10 could contain just one housing components, and not necessarily a top and bottom of component.

A lighting set is primarily characterized by a light emitting direction 200 or plane of which a single or plurality of lighting source(s) 29 it comprises will emit light. A lighting set 30 may be affixed to one or more lighting surfaces 90. A lighting set 30 may have one or more colors of light emitted among the lighting sources 29 comprising each lighting set 30. In a preferred embodiment, each of the lighting sources 29 of either given lighting set 30 emit the same color, or nearly the same color, as defined by the light wavelength, or range of wavelength. In another embodiment, at least two of the lighting sets will emit a different color of light, collectively as the group of lighting sources 29 comprising each lighting set 30.

One or more surfaces of the lighting apparatus 10 may be used as a lighting surface 90 for the purpose of emitting a single or plurality of lighting sources 29 comprising a lighting set 30 in a designated direction. A first lighting set 31 may be comprised of multiple first lighting sources 20, such as LEDs 23 affixed to at least one lighting surface 90, collectively emitting light in one direction or plane.

A single or plurality of first lighting sources 20 collectively comprise the first lighting set 31. In a preferred embodiment, the first lighting sources 20 are LEDs 23, light emitting diodes. The first lighting sources 20 may be configured in an LED array 21. Similarly, a single or plurality of second lighting sources 25 comprising the second lighting set 41 may also be configured in an LED strip, or LED array 21. One or more arrays 21 may comprise any given lighting set 30.

FIG. 1B is a top view of the lighting apparatus 10. A center cylinder component 51 has a central cavity forming an aperture shape. The central cavity serves as a main wire channel 61. The center cylinder component 51 is part of a top housing component 42 in a preferred embodiment. The outer surface of the center cylinder component 51 faces the second lens 100.

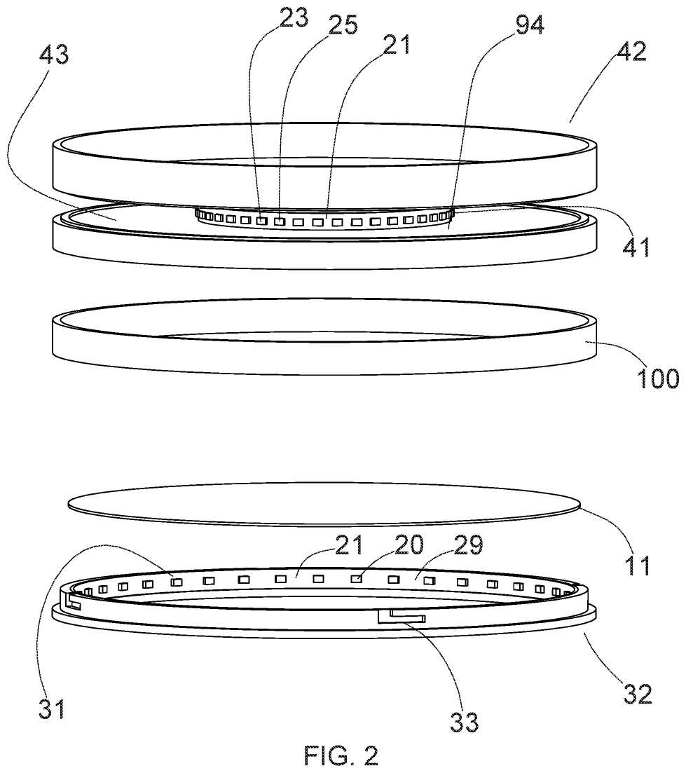

FIG. 2 shows an exploded view of the lighting apparatus 10 in a preferred embodiment. In at least some implementations the lighting apparatus 10 contains two housing components. A bottom housing component 31 attaches to the top housing component 42 to hold the apparatus 10 and its contents together. While it is useful for manufacturing purposes to separate the lighting apparatus 10 into two components, there are practical advantages from the user's perspective as well. When the lighting sets, at least the first lighting set 31 and the second lighting set 41 are primarily used to emit different perceived light colors from one another, or are used in differing frequency, replacing either of the housing components, or the LED light sources 23 comprising the lighting sets within them will be more accessible if configured in two separate housing components. If one housing component is used, as may be the case in at least some examples, it may not be possible to replace the lights at all without altering the apparatus itself. Separating the two lighting sets and surfaces allows for easier replacement of either or both sets. As opposed to replacing both lighting sets, if this is not necessary, separating these two components allows for just one to be replaced, as needed, saving money and energy.

FIG. 2 further illustrates the second lighting set 41 affixed to a second lighting surface A 93. As explained above, it is also possible to utilize other surfaces of the top housing component 42 as a lighting surface 90 such as second lighting surface B 94 in addition to a second lighting source C 95. The number of second lighting sources 25 used to formulate the second lighting set 41 may vary depending on the desired lighting outcome. The upper limit of second lighting sources 25 is primarily limited by the surface area of the top housing component 42 available.

The bottom component 32 comprises the flat circular first lens 11 that can transmit light, in at least some examples. Said lens 11 can be a separate removable component, or attached to the bottom housing component 32. In at least one example there is no top surface of the bottom component 32 but rather an open cavity shaped as a circular aperture formed by the bottom ring of the bottom component 32. The shape of the components, and described top and bottom surfaces may change as the shape of the apparatus 10 may change. In some examples, the apparatus 10, comprising the housing components and the collective lighting apparatus, may be shaped as a triangle, oval, square, etc. In one example, there is a top surface of the bottom component which may serve as a lighting surface.

The outer circumference of the circular top component 42 is comprised of a second lens 100 in between the housing for transmitting light, in at least one example. In some examples the top component 42 could have a different base shape. In another example, the cylindrical inner surface could be another shape, not limited to a cylindrical shape. The size of the cylindrical component 51 or similar shape can also change, moving a second lighting set 41 closer or further from a second lens 100. The shape of the inner surface will effectively serve as a lighting surface 90. Preferably, the inner surface will be raised such that it has a wall, so that at least one lighting set 30 can be affixed to the surface, though the lighting set could use the other lighting surfaces 90 for all of its second lighting sources 25.

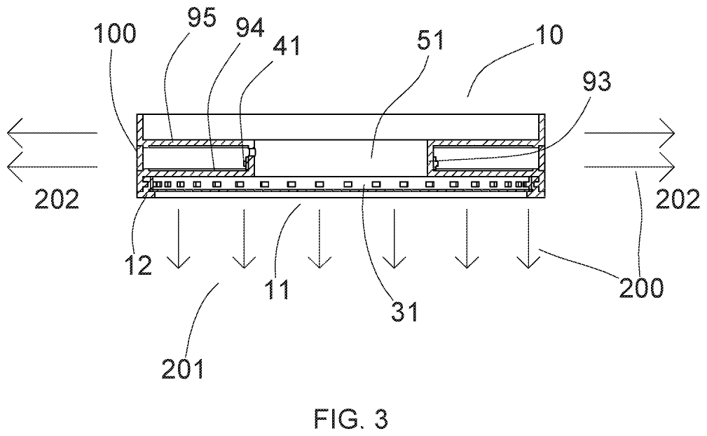

FIG. 3 illustrates a cross-sectional view of the lighting apparatus 10 in a preferred embodiment. The illustration shows clearly how a light interference separator 43 distinguishes at least two lighting sets 30 and eliminates, or substantially minimizes light interference between the two lighting sets within the apparatus 10. The arrows pointing down demonstrate a first light emitting direction 201 of the first lighting set 31. When affixed to a ceiling or ceiling apparatus, such as a ceiling fan, the first light emitting direction 201 is primarily perpendicular to the floor, though the light may illuminate a space more broadly. The arrows pointing left to right demonstrate a second light emitting direction 202 of the second lighting set 41. When affixed to a ceiling or ceiling apparatus, such as a ceiling fan, the second light emitting direction 202 is primarily parallel to the floor, though the light may illuminate a space more broadly.

In a preferred embodiment, the second light emitting direction 202 will emit a UVC light created when the second lighting set 41 is powered ON, and the second lighting sources 25 are primarily UVC LEDs. In said embodiment, the first light emitting direction 201 will a emit a non-UVC light created when the first lighting set 31 is powered ON, and the first lighting sources 20 are primarily non-UVC LEDs which are configured to emit some variation of a white light.

In a preferred embodiment, the second lighting set 41 is configured to emit light outward horizontally in the second light emitting direction 202, parallel to the floor of a room, and the first lighting set 31 is configured to emit light downward in the first light emitting direction 201, perpendicular to the floor. One skilled in the art will know that while a first lighting set 31 is emitting light downward, it still may illuminate an entire room. In an example, the second lighting set 41 emits an ultraviolet light, specifically a UV-C light, characterized by a wavelength of 200-280 nm. Due to the non-intersecting lighting sets affixed to the first and second lighting surfaces 90, the light emitted from both sets will have minimal interference which could otherwise change the amplitude or even the perceived color as a function of constructive interference and destructive interference.

FIG. 3 further shows light emitting from the lighting apparatus in two different directions, in a preferred embodiment, such that interference between the light sources 29 is minimized or absent as a result of the light emitting direction enabled due to, at least in part, the lighting surfaces 30 used for each respective set 30 and the corresponding lens configuration.

In a preferred embodiment the outer surface of the center cylinder component 51 serves as a second lighting surface A 93. The second lighting set 41 is affixed to at least the second lighting surface A 93 in a preferred embodiment. In at least some examples, the second lighting set 41 may have a plurality of second lighting source(s) 25 which comprise the second lighting set 41 affixed to second lighting surface C 95. In at least some examples, the second lighting source(s) 25 may not be affixed to second lighting surface A 93 but only second lighting surface C 95. In yet more examples, the second lighting source(s) 25 comprising the second lighting set 41 may be affixed to a second lighting surface B 94 in addition to, in combination with, or instead of the second lighting surface A 93 and second lighting surface C 95. The lighting surface 90 combinations for the second lighting set 41 are just some examples in some implementations of the arrangements that may be used as a base for one or more lighting sources comprising the second lighting set 41.

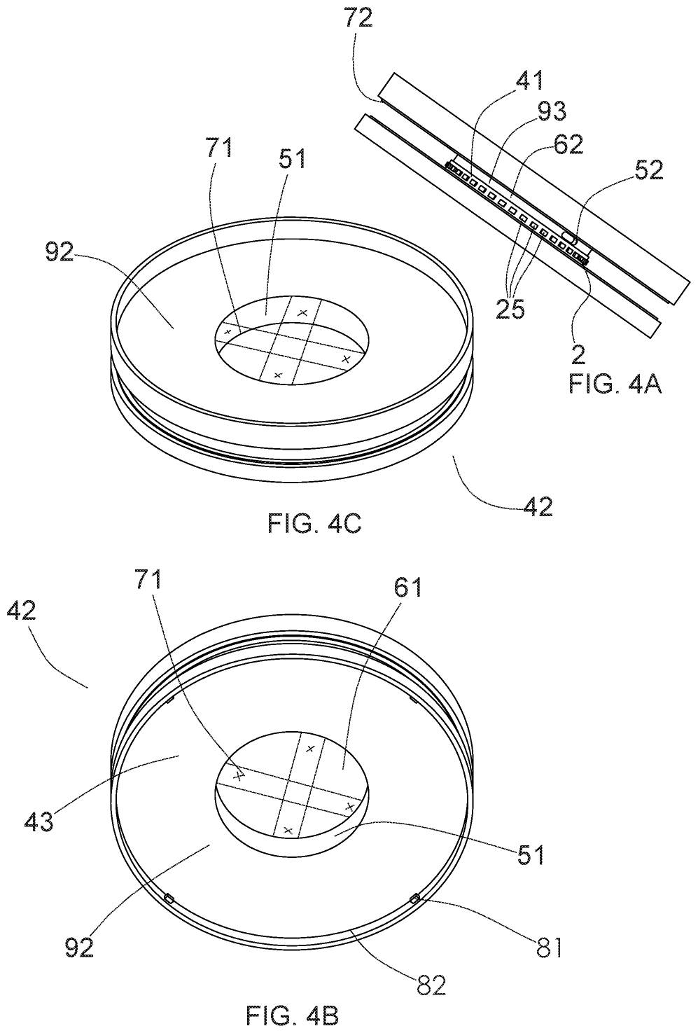

FIG. 4A is a side view of the top housing component 42. The center cylindrical component 51, in a preferred embodiment is useful at least as the lighting surface 90 for the second lighting set 41. The outer surface facing the second lens 100 can be used as a lighting surface 90. There is a small oval shaped hole that may be used as a secondary wire channel 52 into the main wire channel. The hole can be different shapes in other examples. The apparatus 10 does not necessarily need to use the main wire channel 61 in order to pass through the wires in some embodiments. In at least some examples, the channel can also be created in other surfaces of the top housing component 42, such as the second lighting surface C 95. The second lighting sources 25 aligned in a strip circuit board 22, as a single LED array 21, or multiple strip circuit boards 22, otherwise known as LED strips 22, collectively formulate a second lighting set 41. In the illustration, the second lighting set 41 is affixed to the second lighting surface A 93. The second lighting set 41 may be glued on to the lighting surface or be affixed to the lighting surface with other adhesives or attachment mechanisms.

The lighting surfaces 90 of the top housing component 42 will preferably have enough surface area to affix a preferred number of LEDs 23 in one or more LED arrays 22 comprising a second lighting set 41 to emit a preferred level of light limited by the collective amount of surface area. The top component 42 can have more than one lighting surface 90, such as a bottom surface or a top surface in the general shape of the housing component. More than one lighting surface 90 in the top housing component can be used as a base for the second lighting set 41.

The LEDs 23 can be spaced along the LED printed circuit board 22 and the arrays of LED printed circuit board 21 can be spaced along a lighting surface 90 to substantially fill a space along a length of the lighting surface 90. The spacing of the LEDs 23 can be determined based on, for example, the light distribution of each LED 23 and the number of LEDs 23. The spacing of the LEDs 23 can be chosen so that light output by the LEDs 23 is uniform or non-uniform along a length of a first lens 11 and a second lens 200. In addition to spacing the LEDs 23 as described above, the LEDs 23 nearer one or both ends of the LED-based light 10 can be configured to output relatively more light than the other LEDs 23. For instance, LEDs 34 nearer one or both ends of the LED-based light 10 can have a higher light output capacity and can be provided with more power during operation.

FIG. 4B illustrates an example implementation of the light interference separator 43 facing the first lens 11. A single or plurality of attachment mate components 81 in said examples are under the light interference separator 43 on the inside surface of an attachment ring 82. In some implementations the attachment ring 82 could have grooves that enable it to attach to a bottom housing component 32 counterpart with similar grooves in the bottom housing component 32, as an alternative mating option. In a preferred embodiment the attachment mate components 81 mate with a plurality of attachment grooves 33 on the bottom housing component. The central aperture created by the central cylinder component 5, the central aperture also called the main wire channel 61, is shown to enable a power supply from the first lighting set 31 affixed to the bottom housing component 32 to pass through to the main driver. The main driver which distributes power received from a power supply to the LED arrays 21 and associated lighting sources may rest on the top-outer surface of the second lighting surface C 95, parallel to the ceiling, or the driver may be part of another apparatus, such as a ceiling fan. In such an example, the lighting apparatus would mount to the ceiling fan, or similar apparatus, and the wires requiring power supply will connect to a driver distributing power from the ceiling fan, and the associated power supply.

The light interference separator 43 will prevent, or substantially minimize interference between the lighting sets 30. In a preferred embodiment, the light interference separator 43 is a round bottom surface of the top housing component 42. The top side of the light interference separator 43, also a second lighting surface B 94, facing the ceiling, may concurrently serve as an additional lighting surface 90 for the second lighting set 41. Similarly, the bottom side of the light interference separator 43, facing the floor, also serving as a first lighting surface B 92 may concurrently serve as an additional lighting surface for the first lighting set 31. In at least some examples, the light interference separator 43 may be part of the bottom housing component 32.

In some implementations the light interference separator 43 may not necessarily be part of the top housing component 42, but rather could be an additional material, such as a reflector sheet which serves the purpose of separating the light emitted from each of the lighting sets 30

In at least some examples, the light interference separator 43 may have additional materials with reflective properties attached to either side to facilitate transmission of light through each respective lens. The light emitted from the lighting source(s) 29 in each set 30 will be transmitted more effectively when reflection from the lens(s) back towards the lighting apparatus 10 is minimized or re-reflected back. Additional components or materials may be added to the lighting apparatus 10 to better transmit light to the surrounding space. One skilled in the art will recognize that there are numerous ways to influence light transmission.

The bottom housing component 32 can attach to the top housing component 42 of the lighting apparatus 10 in a number of ways. In a preferred embodiment, the attachment groove 33 in the bottom housing component 32 mates with the attachment mate component 81. The bottom housing component 32 and top component 42 could also clipped together, in an example, or be screwed, or welded together, as just a few examples.

The attachment groove 33 on the bottom housing component 32 mates with an attachment mate component 81 on the top housing component 42. In a preferred embodiment the attachment mate components 81 can fit into each of the attachment grooves 33 and slide to hold the two housing components together. In other examples, other attachment configurations may be used to have the similar effect of attaching the top and bottom housing components, or more components, if a plurality of housing components comprise the apparatus 10.

FIG. 4C illustrates a preferred example of a mounting mechanism 71. In a preferred implementation, the mounting mechanism 71 would be the attachment point to a ceiling fan or a ceiling fan motor. In at least some examples the mounting mechanism 71 will attach to a ceiling fan with screws, aligned with similarly spaced screw receptors in the ceiling fan. Similarly, screws may be used to attach the mounting mechanism 71 to a ceiling fan motor.

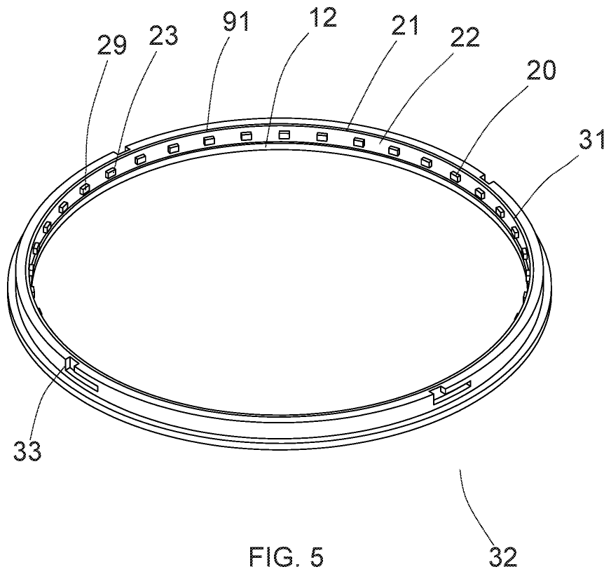

FIG. 5 shows the first lighting set 31 on a first lighting surface A 91, in a preferred embodiment. The lighting set 31 is made up of LED lights on a printed circuit board (strip circuit board) 22. Depending on the size of the fixture, the surface, or the desired light emitting output, the number of arrays 21 and subsequent LEDs 23 lighting sources 29 may increase or decrease. The number of LED's 23 is primarily limited by the amount of surface area available for the LEDs 23 to be attached to. The lighting surface A 91 is the inner surface of the bottom housing component 32 shaped in a ring. In at least one embodiment, a top surface of the bottom housing component 32, forming the shape of a circle, or configured to for the shape of the housing base, if included, may also serve as a lighting surface 90 for the first lighting set 31. Similarly, the bottom surface facing the first lens 11 of the top housing component 42 may serve as a lighting surface 90 for the first lighting set 31, in at least one example. More specifically the inner surface faces another portion of the inner surface of the bottom housing component 32.

The illustrated surface labeled first lighting surface A 91, in one example, is the inner surface of the bottom housing component 32. The first lighting set 31 can be affixed to this inner surface. In one example, an LED strip 21 comprises all the light sources 29 forming the first lighting set 31. The lighting set 31 will emit light outwards towards the first lens 11. The light emitted may be reflected or manipulated when emitted against a lens with any type of pattern or with a separate transmission element added to efficiently distribute light across the plane in the first light emitting direction 201. Similarly, the same can occur for the second lens 100 emitting light in the second light emitting direction 202.

In a preferred embodiment, the lighting sources 20 are LEDs made of the same material composition such that they emit the same color, preferably a variation of white. The lens ridge 12 is elongated such that the first lens 11 may rest on the ridge without sliding through the bottom housing component 32. The light emitting from all the lighting sources 20 will be transmitted through the first lens 11 when the circuit board 22 receives power from a drive circuit board connected to a power supply.

One of the advantages of having at least two distinguished lighting surfaces 90 as a base(s) for at least two lighting sets 30 emitting light in at least two different directions 200, such as a first light emitting direction 201 and a second light emitting direction 202, is that the light(s) collectively emitted from any given set 30, has little to no interference with the lighting source(s) 29 emitting from the other lighting set(s) 30. By minimizing the interference between the lights emitted from each of the lighting sets 30 , the integrity of each of the wavelengths and the corresponding perceived color and brightness remains as though any given set 30 was operable with the characteristics intended. Separating the lighting sets 30 through the lighting surfaces such that the light emitted does not interfere within the lighting apparatus 10, will minimize both the constrictive and destructive interference that may effect light waves emitted.

Yet another advantage of having two separate lighting sets 30 affixed to two or more surfaces 29 emitting light in different directions is the ability to create the desired ambience through the color and intensity of a first lighting set 31, without compromising the properties of the second lighting set, which when emitting UV-C light can combat against bacteria and pathogens at the same time, as discussed above. There may be cases where a user does not wish to primarily see the UV-C light and prefers the more traditional ambience created by LED lights 23, such as those visible on the UV spectrum in shades of yellows or whites.

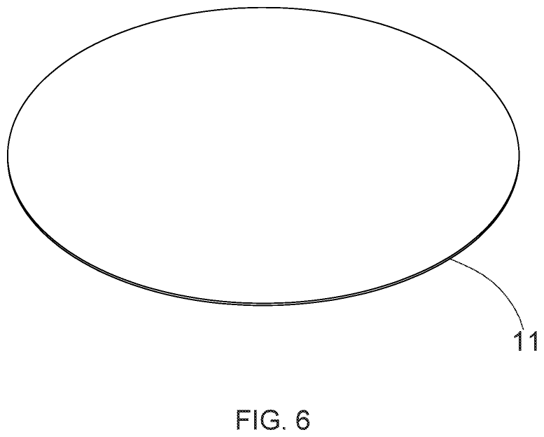

FIG. 6 shows the first lens 11. In a preferred embodiment first lens 11 has a flat circular shape and transmits light emitted from the first lighting set 31 in a direction perpendicular to the floor of a room, the first light emitting direction 201, when the lighting apparatus 10 is affixed to a ceiling or ceiling mounted device, such as a ceiling fan. In a preferred embodiment, the circular shaped first lens 11 is primarily flat and rests on a first lens ridge 12. In other examples, the lens may be a square, triangle, oval, rectangle, or a variety of other shapes according to the shape of the apparatus 10 and design preference. Light emitted from the plurality of first lighting sources 20 will transmit through the first lens 11.

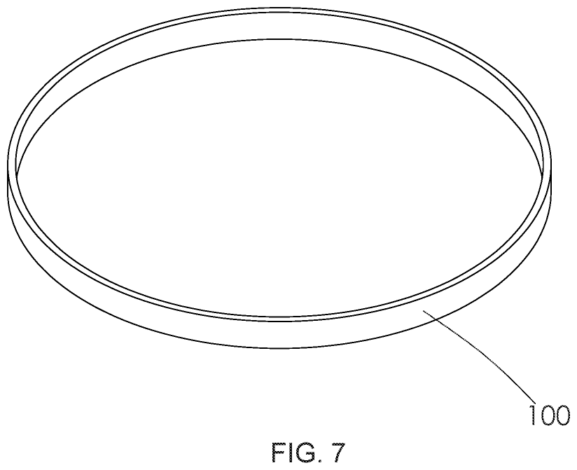

FIG. 7 shows the second lens 100. The second lens 100 is primarily used to transmit the light emitted from second lighting set 41. In a preferred embodiment, the second lens 100 has a cylindrical shape with an inner and outer diameter so to create the shape of a central aperture. The second lens 100 transmits light collectively emitted from the second lighting set 41 in the second light emitting direction 202. The second lens 100 is affixed to the top housing component 42 on a second lens mounting surface 72. The second lens 100 may be glued to the second lens mounting surface 72 or rest into a ridge created, in some examples.

Constructive and destructive interference may occur when light or sound waves pass through each other, occupying the same point. The individual waves will add together, known as superposition, so that a new wavefront is created with a higher or lower amplitude than the original waves. Superposition of waves leads to interference. There are two types of interference, constructive and destructive, designated by the net result of the waves crossing. Interference can simply be explained as a reaction between waves which cross over or meet each other while traveling in the same medium. Destructive interference is characterized by the added two waves canceling out, while constructive interference refers to the amplitude of the waves added to create a larger amplitude.

The amplitude of a light wave refers to its brightness. Another type of interference can occur in the form of the perceived color of the light when multiple light waves in close proximity emit light of varying colors. As a simplified explanation, the human eye will take the overlap of wavelengths of incoming light against the response function of the S, M, and L photoreceptors to interpret a color. In the lighting apparatus 10, light is reflected throughout a space so surely some interference will occur, but changing the direction 200, or planes through which the light will primarily emit from the original source will greatly minimize the interference of the other lighting set(s) 30 within the lighting apparatus 10.

Integral to any lighting fixture is the ability to transmit light. Light typically passes through a lens component which is affixed to one or more housing components. Different manufacturing methods may be applied to the LED 23 or different components may be added to the lighting apparatus 10 to better distribute the light emitted from the lighting source 29 throughout the room. Depending on the refractive index of the lens, transmittance of the light may be minimized depending on the materials and coating of the lens. Optical surfaces, such as a lens, may be coated with various materials to reduce reflection back towards the lighting source, and increase transmission of light away from the lighting source. In some implementations, the lens may have small imperfections, such as pinholes, scratches, bubbles, etc. to scatter light. A reflective layer or material may also be added facing the lens such that any light reflected by the lens may be re-reflected back out for transmission.

In at least one implementation, the first lighting set 31 has a reflective layer affixed to the outer-bottom surface of the top component so that light reflected back from the lens can be re-reflected back for more transmission.

Each of the lighting sets may have one or more arrays 21 of LED lights 23. Each array 21 may have a single or plurality of LEDs 23. An array 21 of LEDs 23 could be on a printed circuit board strip 22 or on a chip affixed to circuitry. Multiple arrays 21 and their corresponding circuitry can be combined to one another and controlled via the same power supply and driver circuit board. The driver circuit board connects to the power supply to control current to a lighting set and the LED arrays 21 comprising each lighting set. A top housing component 42 may also contain the driver circuit board which receives a power supply may be part of another apparatus, such as a ceiling fan.

A single LED array 21 or a plurality of LED arrays 21 may be configured to form an LED lighting set. The arrays 21 may be organized to cover a lighting surface 90 in a pattern that emits light according to preference. An LED array 21 may be connected to another LED array 21 and controlled by the same driver or may be a standalone array 21 that receives current from a separate controller. An LED strip light 22 is a flexible circuit board populated by surface mounted LEDs 23 and other components that usually comes with an adhesive backing. Multiple LED strips 22 can connect to another central circuit which is connected to a power supply. The central circuit can have a communication protocol with a remote or app device which can control each individual LED strip 22.

The orientation, number and spacing of the LEDs 23 can be a function of a length of the LED-based light 10, a desired lumen output of the LED-based light 10, the wattage of the LEDs 23 a desired light distribution for the LED-based light 10 and/or the viewing angle of the LEDs 23.

The LEDs 23, for example, may be selectively driven to vary the intensity of light illuminating along the length of the LED-based light 10. For instance, the LEDs 23 along one half of the LED-based light 10 could be driven in an ON state to emit light, and the LEDs 23 along the other half of the LED-based light 10 could be driven in an OFF state, resulting in light being illuminated from only half of the LED-based light 10.

The intensity of light, or the brightness, is controlled by the amount of current provided. It is suggested that the wattage of the driver be greater than the sum of the wattage of each LED 23 in an array 22. An LED light 23, which is ultimately perceived as a color, is formed when bringing P-Type (+) and N-Type (−) semiconductors together that form a PN Junction. In an LED 23, a p-n junction emits light when electric current flows through it. Electrons cross from the n-region and recombine with the holes existing in the p-region. Free electrons are in the conduction band of energy levels, while holes are in the valence energy band. Thus, the energy level of the holes is lower than the energy levels of the electrons. Some portion of the energy must be dissipated to recombine the electrons and the holes. When a forward bias (or voltage) is applied, electrons in the n-type region are pushed toward the p-type region and, likewise, holes in the p-type material are pushed in the opposite direction (since they are positively charged) toward the n-type material. At the junction between the p-type and n-type materials, the electrons and holes will recombine and each recombination event will produce a quantum of energy that is an intrinsic property of the semiconductor where the recombination occurs. This energy is emitted in the form of heat and light.

The electrons dissipate energy in the form of heat for silicon and germanium diodes, but in gallium arsenide phosphide (GaAsP) and gallium phosphide (GaP) semiconductors, the electrons dissipate energy by emitting photons. If the semiconductor is translucent, the junction becomes the source of light, thus becoming a light-emitting diode. The precise wavelength (color) can be tuned by altering the composition of the light-emitting, or active, region. LEDs are comprised of compound semiconductor materials, which are made up of elements from group III and group V of the periodic table (these are known as III-V materials). Examples of III-V materials commonly used to make LEDs 23 are gallium arsenide (GaAs) and gallium phosphide (GaP).

When a light emitting diode is electrically connected, electrons start moving at the junction of the N-type and P-type semiconductors within the diode. When there is a jump over of electrons at the p-n junction, the electron loses a portion of its energy. In regular diodes this energy loss is in the form of heat. However, in LEDs 23 the specific type of N and P conductors produce photons (light) instead of heat. The amount of energy lost defines the color of light produce

The wavelength of the light emitted, and thus its color, depends on the band gap energy of the materials forming the p-n junction. LEDs 23 are usually built on an n-type substrate, with an electrode attached to the p-type layer deposited on its surface. P-type substrates, while less common, occur as well. Many commercial LEDs 23, especially GaN/InGaN, also use sapphire substrate. The term “band gap” refers to the energy difference between the top of the valence band and the bottom of the conduction band. Electrons are able to jump from one band to another. However, in order for an electron to jump from a valence band to a conduction band, it requires a specific minimum amount of energy for the transition. The required energy differs with different materials. The color of the light (corresponding to the energy of the photons) is determined by the energy required for electrons to cross the band gap of the semiconductor.

The color emitted by each LED 23, perceived by the human eye, and the subsequent wavelength, is also attributable in large part to the material used in the semi-conductor of the LED 23, as explained above, and specifically the doping used. The material primarily used for UV LEDs 23 is gallium nitride/aluminum gallium nitride (GaN/AIGaN) at wavelengths 360 nm or longer. Shorter wavelengths utilize proprietary materials. LEDs use materials that can handle the necessary levels of electricity, heat, and humidity. High-brightness red and amber LEDs 23use the aluminum indium gallium phosphide (AlInGaP) material system. Blue, green and cyan LEDs 23 use the indium gallium nitride (InGaN) system. Together, AlInGaP and InGaN cover almost the entire light spectrum, with a gap at green-yellow and yellow. One method of achieving a larger spectrum of colors is to mix different colors of LEDs 23 in the same device. Combining red, green, and blue LEDs 23 in a single LED device, such as a lighting apparatus 10 or multi-chip LED, and controlling their relative intensities can produce millions of colors. Additionally, combining red, green, and blue in equal amounts produces white light.

The process of creating chemical impurities within the semiconductor wafer is known as doping. This requires very little energy and is what LED 23 energy efficiency is based on. When electricity jumps from the positively charged area to a negatively material the jump releases energy that in turn produces light. The wafer, also called a slice or substrate, is a thin slice of semiconductor material that is composed of various chemicals that serve to transfer electrons from a high-density area to a lower density area.

In a preferred embodiment, at least one of the lighting sets 30 will primarily emit UV light. An LED 23 may be configured to emit UV light. In at least some implementations, the second lighting set 41 will emit UV light.

UV light covers a wavelength spectrum from 100 to 400 nm and is subdivided into three regions by wavelength: UVA (320 to 400 nm), UVB (280 to 320 nm), and UVC (100 to 280 nm). Among them, UVC has the strongest germicidal effect and is widely used in the form of mercury lamps to inactivate microorganisms. However, UV mercury lamps have several critical limitations. First, UV lamps are fragile and thus present a risk of mercury leakage through breakage when subjected to any shock. Also, the warm-up time is long and, moreover, cannot exhibit maximum efficacy at low temperatures according to an earlier study. Due to these critical weaknesses of mercury lamps, UV light-emitting diode (UV-LED) technology has been developed recently as an alternative. In the case of disinfection, the optimum wavelength is in the region of 200 nm to 270 nm, with germicidal efficacy falling exponentially with longer wavelengths. UVC LEDs offer considerable advantages over the traditionally used mercury lamps, notably they contain no hazardous material, can be switched ON/OFF instantaneously and without cycling limitation, have lower heat consumption, directed heat extraction, and are more durable.

UV-C photons penetrate cells and damage the nucleic acid, rendering them incapable of reproduction, or microbiologically inactive. This process occurs in nature; the sun emits UV rays that perform this way. In other words, UV light produces electromagnetic energy that can destroy the ability of microorganisms to reproduce and by causing photo-chemical reactions in nucleic acids (DNA & RNA). The ultraviolet energy triggers the formation of specific thymine or cytosine dimers in DNA and uracil dimers in RNA, which causes inactivation of microbes by causing mutations and/or cell death and failure to reproduce. UV represents wavelengths that fall between visible light and x-ray on the electromagnetic spectrum.

Germicidal ultraviolet light, typically at 254 nm, is effective in this context but can be a health hazard to skin and eyes. By contrast, far-UVC light (207-222 nm) efficiently kills pathogens potentially without harm to exposed human tissues. In a preferred embodiment, the UVC light emitted from one lighting set will be a far UVC light in one or more arrays 21 or lighting sources comprising a lighting set.

UV-C rays have short wavelength, and therefore high energy relative to the more traditional color spectrum, and thus are capable of killing bacteria and viruses, also called pathogens. Ultraviolet (UV) light destroys the molecular bonds that hold together the DNA of viruses and bacteria. Ultraviolet (UV) light is characterized by a wavelength range of 100-400 nm, but UV-C (or UV) specifically has demonstrated properties that effectively combat pathogens. We may use these interchangeably throughout as different UVC lights may be used to serve the purpose of sterilizing air in a room.

In a preferred embodiment, at least one of the lighting sets 30, and all the lighting sources 29 within each set 30, preferably LEDs 23, is configured to emit ultraviolet light, specifically UV-C light, classified by a wavelength on the electromagnetic spectrum of about 200-280 nanometers (nm). A wavelength of about 220-260 is most preferable. In a preferred embodiment, the lighting set comprising UV-C emitting lighting sources 29, is directed left to right, wall-to wall in a room, or parallel to the floor of a room, in the second light emitting direction 202.

In yet another embodiment, one or more lighting sets 30 may contain more than one color lighting source(s) 29 within each lighting set 30, at least one of which emits UVC light. In said embodiment, the UVC light source 29 shares one or more surfaces 90 with the other lighting sources 29 of the lighting set 30, though it could have its own lighting surface 90. In said embodiment, the UVC lighting source could have a different power control from the other lighting sources such that it could be switched ON while the other lighting source(s) is switched OFF. To make clear, a lighting set 30 that contains both a UVC light and at least one other colored light in a given set 30, will still emit light in the same direction as the other lighting sources within the lighting set. In said example, the lighting apparatus 10 could emit UVC light in both directions of each of the lighting sets. Still further, the lighting set 30 could emit UVC light 29 perpendicular to the floor 201, while another lighting set 30 is emitting non-UVC wavelength light parallel to the floor 202.

In a preferred embodiment, and as described above, the addition of a UV light, or UV led light is the electromagnetic wavelength primarily emitted from at least one of the lighting sets affixed to at least one of the lighting surfaces. In at least one example, the UV light is emitted from the second lighting set 41, illuminating in a direction parallel to the floor 202.

In said example, it is important for there to be minimal, and preferably no interference from another lighting set 30, particularly if the other lighting set is not emitting UV light in order to best achieve the effect of disinfecting the surrounding air, proximate to the UV-C lighting set 41. The ability for a lighting apparatus 10 to direct different light sources in different directions, as achieved through the separation and configuration of the lighting surfaces and lens' where the light transmits through, enables a user to benefit from the ambience created by a traditional light without disruption or interference from the other lighting set. Still further, this ambience can be maintained while at least one secondary lighting set 41, emitting a UV-C light in the preferred example, can retain its properties which help sterilize the air in a room without, or with minimal interference.

In a preferred embodiment, the composition of at least some of materials used for these semiconductors in the first lighting set 31, will be different from at least some of the materials used for the semiconductors of the second lighting set 41.

Each of the lighting sets 30 may have a combination of LEDs 23 configured to project a particular color, intensity, or brightness. In a preferred embodiment, the LEDs 23 of each lighting set are configured to emit different perceived light color from the other, as characterized by the wavelength, or wavelength range of the light(s) with an appropriate allowable variance.

In a preferred embodiment, the second lighting set 41 emits a different wavelength than the first lighting set 31, appearing to the human eye as a different color. Each of the LEDs 23 in any given lighting set 30 may be configured to emit a different wavelength, however.

In at least one embodiment, light emitted from both of the LED lighting sets 30 each affixed to one or more of the lighting surfaces 90 is identical or near identical, emitting the same or roughly the same wavelengths. In another embodiment, the wavelength emitted from at least two of the lighting sets 30 falls within the UVC range.

In at least one example, the first lighting set 31 has at least two lighting surfaces 30, a first lighting surface A 91 and a first lighting surface B 92. In said example, one of the lighting surfaces 90 has UVC LEDs 23 affixed to it while the other lighting surface 90 has traditional white light emitting from the LEDs 23 affixed said lighting surface 90. Both the lighting surfaces 90, and the lighting sources 29 affixed to them will emit light in the same first direction 201 through the first lens 11.

In another example, the second lighting set 41 has at least two lighting surfaces 90. In said example, one of the lighting surfaces 90 has UVC LEDs affixed to it while the other lighting surface has traditional white light emitting from the LED 23 Ds affixed said lighting surface 90. Both the lighting surfaces 90, and the lighting sources 29 affixed to them will emit light in the same second light direction 202 through the second lens 100.

In yet another example, both the first and second lighting sets 30 have at least two lighting surfaces 90. In said example, one of the lighting surfaces 90 of each set has UVC LEDs 23 affixed to it while the other lighting surface 90 of each set 30 has traditional white light emitting from the LEDs 23 affixed said lighting surface 90. Both the lighting surfaces 90, and the lighting surfaces 90 affixed to them will emit light in the same direction through the lens.

In a preferred embodiment, the lighting apparatus 10 will be affixed to a ceiling fan. When a UV-C light is emitted as the preferred wavelength/frequency for the second lighting set 41 emitting light in a direction 202 parallel to the floor, air passing the fan could be exposed to the sterilizing properties of UV-C light, when the light is switched ON, without, or with minimal sterilizing efficacy compromised by the interference, constructive or destructive, of the first lighting set or any other lighting set in the apparatus. In further implementations air could pass a UVC light source in the first lighting set 31 in addition to or instead of the UVC light source in the second lighting set 41. The sterilizing effects of the UV light described above may vary with the number of UV lighting sources comprising each lighting set, the power or current they receive, and the properties of the lens used to transmit the light, as just a few examples of factors that may impact sterilizing efficacy. The lighting apparatus 10 may be configured to have a range of UV LED arrays 21 within the second lighting set 41, with a range of lighting sources, or diodes, in each LED array 21.

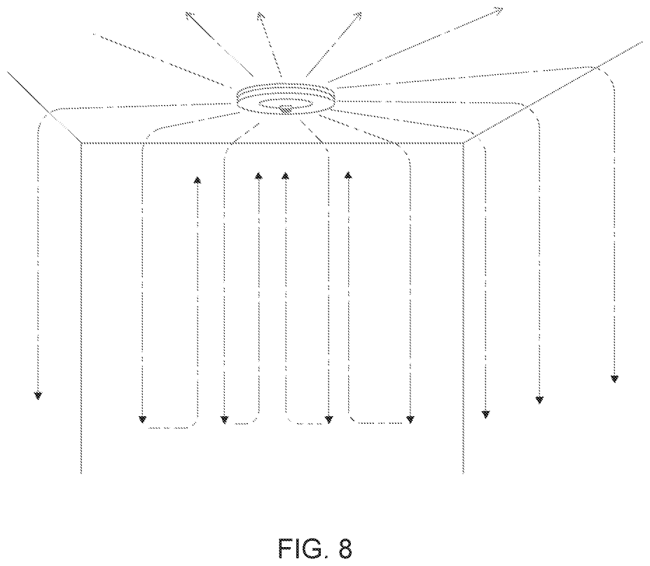

FIG. 8 depicts a method for sterilizing air in a room with the lighting apparatus 10 affixed to a ceiling fan apparatus. In a preferred embodiment, the lighting apparatus 10 is mounted to a ceiling fan that uses discs instead of pitched blades, which are affixed to a central axis powered by a drive system, such that when rotated, air is drawn up from below the ceiling fan and pushed outward along the discs to create air circulation. In yet another embodiment, according to an example, pitched blades may be used on the ceiling fan apparatus, common in most ceiling fans configured to move air. In the preferred embodiment, the rotation of the discs creates air circulation. When the second lighting set 41 affixed to the second lighting surface(s) 93/94/95 is powered ON, the air drawn from the ceiling fan will pass the light emitted from the second lighting set 41. In said embodiment, this light is a UV-C light characterized by wavelength of 200-280 nanometers.

To reiterate, the advantage of this is that air circulating will pass the UVC light emitted with the properties described above that can effectively disinfect the air in passing. When air is circulating for a sufficient amount of time, the air has a greater likelihood of sterilizing and continuous movement in conjunction with the second lighting set 41 powered ON will continue to sterilize the air. In the preferred embodiment the first lighting set 31 may also be powered ON or OFF, irrespective of the second lights 41 ON/OFF status. In at least one example, the first lighting set 31 could also be a UVC LED 23. Preferably, however, the first lighting set 31 is a traditional LED 23 emitting light perceived by the human eye as some form of white. The lighting apparatus 10 affixed to a ceiling fan apparatus, in some examples, may be configured to change the light intensity, change the speed of the apparatus, or any combination of the two to expose a range of air volume to the UV-C light and disinfect according to the properties of the UV-C.

A power modulation control unit in communication with the plurality of LEDs 23 may be configured to select and energize in response to a data input and thereby cause at least one LED array 21 to emit light and thereby irradiate the subject with non-ultraviolet electromagnetic radiation. Yet another LED array 23 or groups of LED arrays 23 may be controlled separately via another data input whereby the LED arrays 23 comprising a different lighting set, such as a second lighting set 41 may emit light. Each of the LED arrays 23 in both the first lighting set 31 and second lighting set 41 may be dimmed via the power modulation control unit, or the controller.

Each of the plurality of lighting sets 30 may have an individual control address or share a control address such that each lighting set 30 can be operated separately or together, generating multiple lighting outcomes. For example, one lighting outcome could have two or more lighting sets switched ON, or just one lighting set switched ON and the other lighting set switched OFF. Even further, each lighting set may have one or more of the lighting sources dimmable, in one example. In another example, each lighting set, or one or more of the lighting sources comprising each lighting set, may be configured to change colors. Similarly, each of the lighting sources comprising a lighting set may be configured to both change colors and be dimmable.

A central controller can control each lighting set 30 and be further configured to control one or more arrays 21 within a lighting set 30. Some control features of the controller may include the brightness or intensity of the LED array, the whole lighting set 30, the selection of arrays ON/OFF within a lighting set, and the ON or OFF designation of the lighting set 30. In at least one embodiment, the colors of the LED 23 perceived by the human eye, in one or more lighting sets 30, may be controlled by the controller by changing the current passing through one or more LEDs packaged closely. The flexibility of the controller allows the lighting apparatus to create different room ambiance according to the user's preference and permissible through the properties of the light.

The second lighting set 41 can be powered ON or OFF separately or together with the first lighting set 31. The second lighting set can be dimmed separate or together with the first lighting set 31 controlled by the same controlling device or a different controlling device than that which is used to control the first lighting set 31.

What is claimed is:

1. A lighting apparatus comprising:
a plurality of lighting sources,
a plurality of lighting sets,
a plurality of light-emitting directions,
at least one light interference separator,
at least one lens,
a plurality of lighting surfaces,
a housing structure.

2. The apparatus of claim 1 wherein the at least one of the lighting sets is comprised of light emitting diode (LED) lighting sources.

3. The apparatus of claim 2 wherein at least one of the lighting sets is comprised of at least UVC LED lighting sources with a wavelength range of 200 nm to 280 nm on the electromagnetic spectrum.

4. The apparatus of claim 3 wherein at least a first lighting set is comprised of LEDS emitting light perceived as white light

5. The apparatus of claim 4 wherein at least a second lighting sets comprised of UVC LEDs is emitting light in a direction parallel to the floor when mounted on a ceiling or ceiling apparatus.

6. The apparatus of claim 4 wherein a second lighting set comprised of at least UVC LED lighting sources will emit UVC light through a second lens

7. The apparatus of claim 5 wherein a second lighting set comprised of at least UVC LED lighting sources will emit UVC light through a second lens and a first lighting set comprised of LEDs will emit non-UVC light in a different direction through a first lens.

8. The apparatus of claim 7 wherein the first lighting set comprised of non-UVC LEDs is emitting light perpendicular to the floor when mounted on a ceiling or ceiling apparatus.

9. The apparatus of claim 7 wherein the housing structure has a top component and a bottom component

10. The apparatus of claim 9 wherein at least a first lighting set is affixed to at least one lighting surface on the bottom housing component

11. The apparatus of claim 10 wherein at least a second lighting set is affixed to at least one lighting surface on the top housing component.

12. The apparatus of claim 11 wherein the light interference separator is part of the top housing component structure.

13. The apparatus of claim 11 wherein the light interference separator is part of the bottom housing component structure.

14. The apparatus of claim 11 wherein the second lighting set affixed to at least one lighting surface on the top housing component is comprised of UVC LED light sources emitting through a second lens.

15. The apparatus of claim 14 operable through a controller which can control at least the brightness of at least a first and second lighting in addition to power at least a first and second lighting set ON or OFF.

16. The apparatus of claim 14 wherein the housing structure top and bottom base has a circular shape such that a flat circular first lens can rest on the bottom housing component and transmit light emitted from the lighting sources of the first lighting set.

17. A method for sterilizing air in a room comprising:
a lighting apparatus comprising:
a plurality of lighting sources,
a plurality of lighting sets wherein at least one of the lighting sets emits a UVC light,
a plurality of light-emitting directions wherein at least one the light-emitting directions of at least one of the lighting sets emitting a UVC light is in a direction parallel to the floor,
at least one light interference separator,
at least one lens,
a plurality of lighting surfaces,
a housing structure,
air sterilization occurring through method steps comprising:
the lighting apparatus with a UVC light emitting in at least a direction parallel to the floor is powered ON, the lighting apparatus is mounted to a ceiling fan device that is mounted on a ceiling such that air circulation caused by the rotation of the ceiling fan blades or discs causes the air to be exposed to the UVC light which has sterilizing qualities, and continuous air circulation thereafter causes additional exposure to the UVC LED lights.

18. The method of claim 17 wherein the lighting apparatus is affixed to ceiling fan apparatus that uses blades to rotate about a central axis to create laminar airflow.

19. The method of claim 18 wherein a first lighting set emits a white light from an LED lighting source perpendicular to the floor through a first lens to maintain a desired ambience.

20. The method of claim 19 wherein both the first lighting set emitting a white light from an LED light source and a second lighting set emitting a UVC light from an LED light source can both operate at the same time.

21. The method of claim 19 wherein both the ceiling fan apparatus and at least a first and second lighting set are controlled by the same controller
